(19) United States
(12) Patent Application Publication
(10) Pub. No.: US 2003/0211464 A1
(43) Pub. Date: Nov. 13, 2003

(54) Method and compositions for drug discovery

(76) Inventor: Charles Pidgeon, Arlington, MA (US)

Correspondence Address: Barnes & Thornburg, 11 South Meridian Street, Indianapolis, IN, 46204, US

(21) Appl. No.: 10/275,142

(22) PCT Filed: May 2, 2001
(86) PCT No.: PCT/US01/14091

(51) Int. Cl. C12Q 1/70; G01N 33/53; G01N 33/543

(52) U.S. Cl. 435/5; 435/7.1; 436/518

(57) ABSTRACT

A drug-binding substrate formed or identified using a drug substance having a predetermined biological activity is used to screen and identify test compounds likely to exhibit the predetermined biological activity. The potential biologically active test compounds are identified by their specific binding to the drug-binding substrates as detected by any of a wide variety of techniques using labeled or unlabeled assay components. In one embodiment a monoclonal antibody raised against a drug substance is used as a drug-binding substrate to identify and isolate test compounds in a natural product extract or a combinatorial chemical library. Preferably the monoclonal antibody is characterized by its ability to bind specifically to at least one other drug substance having the same or similar biological activity as the drug substance against which it was raised. The invention finds use inter alia in drug discovery protocols, in toxicity profiling of drug substances and in assaying commercial natural products.

### FIELD OF THE INVENTION

The present invention relates to a method of screening test compounds to identify those compounds exhibiting a potential biological activity. The method is based on the specific binding of test compounds to drug-binding substrates formed or identified using drug substances known to exhibit the biological activity.

### BACKGROUND AND SUMMARY OF THE INVENTION

Recently there has been a significant effort directed to the definition of screening methods capable of identifying biologically active compounds. The goal of such efforts is to define efficient methodologies for identifying or predicting in vivo biological activity by in vitro test methods without the time investment and expense of costly animal studies. For many years most drug screening methods were based on conventional in vitro biological activity assays. Such bioassays measure the effect of sources of test compounds on the viability or metabolism of a disease-related cell type such as bacteria, fungi, viruses, and tumor cells. For example, β-lactam antibiotics were discovered by testing microbial broths for bacterial growth inhibition in culture tests. Likewise, the antifungal compounds myostatin and amphotericin B were isolated from broths that inhibited growth of yeast in culture tests. Most bioassays have now been replaced as primary screens with more sophisticated, mechanism-based assays, such as enzyme (biochemical) assays and binding assays. Such assays can be designed, and indeed today are designed with view of the need for high throughput capacity requiring that the components and protocols for such assays be robust, simple and amendable to automation.

Binding assays are particularly useful for screening soluble mixtures of test compounds for compounds that bind to, and thus potentially inhibit or activate, target endogenous molecules with resultant therapeutic effect. The target endogenous molecule, usually a macromolecule such as a protein or a nucleic acid, or a derivative or analog thereof, can be affixed or tethered to a solid substrate, such as the sides of microtiter wells, beads, or chromatographic supports. If the target molecule is a receptor, it can be expressed in the membrane of a cell, which can itself be attached to the solid support. The test compounds, typically in solution, are incubated with the immobilized targets, and bound ligands are detected, frequently through an associated colorimetric or fluorescent reaction. Alternatively, test compounds may be mixed with soluble-phase target molecules that are captured using an anti-target antibody. Such binding assays are advantageous as they facilitate the washing and isolation of target-ligand complexes. However, they suffer from several disadvantages, not the least of which is the availability, isolation, and handling of the endogenous target molecules, each of which has unique chemical and physical properties that must be taken into account in assay development. Moreover, such assays are focused on detecting but one quality of the test compounds, that is their ability to specifically bind to the target molecule.

Drug substances possess a plurality of properties that allow them to exhibit their therapeutic effect in the species to which they are administered. Known drug substances not only exhibit binding to therapeutically valuable endogenous biologically functional molecules, they each also possess other properties necessary for in vivo drug functionality, including but not limited to, the so-called “ADME” properties (adsorption, distribution, metabolism and elimination). The set of drugs on the market are thus a unique set of compounds, subsets of which bind to the same target molecules, but all of which necessarily have in common certain properties that allow them to be efficacious therapeutics when administered to a species being treated.

One objective of the present invention is to use a set of drug-binding substrates, for example antibodies, each of said drug-binding substrates being formed using or identified by one or more known drug substances having the same or similar biological activity. The set of drug-binding substrates is used for identifying and purifying test compounds having potential for biological activity. A known drug or set of known drugs having the same or different biological activities can be used to prepare or identify a drug-binding substrate or a corresponding set of drug-binding substrates, each of which are capable of specific binding to the drug from which they are prepared. One or more members of the set of drug-binding substrates can be used alone or in combination with other members of the set to identify and purify compounds from complex mixtures. In one embodiment one or more drug binding substrates, for example monoclonal antibodies, known to specifically bind to two or more drug substances having the same or similar biological activity is used to screen combinatorial libraries, natural product mixtures, or other sources of potential drug candidates, for compounds that exhibit specific binding to said substrates. Test compounds that specifically bind to the drug-binding substrates will be more likely to exhibit the specific binding and ADME properties of the drug substance(s) from which the drug-binding substrates are prepared. Thus, for example, an anti-drug antibody or, more typically, a set of anti-drug antibodies, can be used to find compounds capable of occupying the same or similar “chemical space” as the drug-substance(s) used to form the antibodies. The drug-binding substrates employed in this invention are, therefore, not only predictive of specific binding of a test compound to the drug-targeted endogenous molecules, but they are, as well, selective for test compounds having ADME properties critical for drug functionality.

The present invention is directed to a method of screening a test compound or a set of test compounds for potential biological or chemical activity using a binding assay wherein the drug-binding substrate is other than the endogenous target molecule or a chemically modified form or analog thereof. Instead, the drug-binding substrate is one that is prepared, or empirically determined, to bind specifically to a drug substance, that is, an exogenous compound that is known or has been shown to exhibit a systemic effect by specific binding to one or more structurally related endogenous target molecules in the species to which it is administered or applied. Thus, in accordance with this invention, known drug substances are used as a template for preparing drug-binding substrates, or for identifying non-endogenous drug-binding substrates, which exhibit specific binding affinity for said drug substance and often for other drug substances having the same or similar biological activity.

The drug-binding substrates useful in accordance with the present invention include antibodies raised against drug substances, particularly monoclonal antibodies, phage display libraries, and polymers imprinted molecularly with drug substances. The drug-binding substrates are selected, in one aspect of the invention, to bind to at least a second drug substance having the same or similar biological activity as the drug substance used to prepare or identify the drug-binding substrate. Such cross-reactivity favors the use of such substrates to identify, by specific binding interaction, test compounds that not only have the potential for specific binding to the endogenous target molecule, but have as well the pharmaceutically significant properties that are required for favorable drug absorption, distribution, metabolism and elimination.

Thus, in accordance with one embodiment of the present invention there is provided a method for screening a test compound or a set of test compounds for potential biological activity. The method includes the step of selecting a drug-binding substrate exhibiting specific binding affinity for a drug substance having a biological activity known to derive from specific binding of that drug substance to one or more related endogenous molecules. The drug-binding substrate is contacted with a test solution comprising a test compound, and the existence of specific binding of the test compound to the drug-binding substrate is determined. In one embodiment the drug-binding substrate is a monoclonal antibody raised against a drug substance (or more precisely, a monoclonal antibody raised against an immunogenic drug hapten-carrier conjugate, formed using said drug substances). The monoclonal antibody is preferably one that exhibits specific binding affinity for at least a second drug substance having the same or similar biological activity as the drug substance used to form the antibody.

Drug-binding substrate (antibody) cross-reactivity is key to enablement and implementation of the present invention. Typically the goal in preparing antibodies to specific binding partners to a ligand or analyte has been one of high specificity and minimum cross-reactivity. The present invention, on the other hand, relies on and indeed requires, drug-binding substrate cross-reactivity to identify test compounds having potential for exhibiting the biological activity of the drug substance used to form or identify the drug-binding substrate.

In another embodiment of the invention there is provided a method for identifying or selecting a drug-binding substrate for use in drug discovery testing. The method comprises the steps of contacting potential drug-binding substrates with a solution of a drug substance having a known biological activity deriving from specific binding with one or more related endogenous target molecules. In one embodiment of the invention the substrates exhibiting specific binding affinity to the drug substances are identified and contacted with a solution of a second drug substance exhibiting the same or similar biological activity as the first drug substance. Alternatively, potential drug-binding substrates are contacted with a solution comprising a plurality of drug substances, each having a common biological activity deriving from specific binding to one or more related endogenous molecules. Those substrates which specifically bind to more than one of the drug substances are preferred for use in drug discovery testing in accordance with this invention.

In another embodiment of the present invention there is provided a kit and/or composition for analysis or screening of compounds for biological activity. The kit or composition comprises at least first and second drug-binding substrates wherein said first substrate exhibits a specific binding affinity to a first drug substance having a known biological activity deriving from its specific binding to one or more related endogenous molecules, and said second drug-binding substrate exhibits specific binding affinity to the same drug substance or to another drug substance having the same or similar biological activity as the first drug substance. In one such embodiment of the invention the drug-binding substrates are monoclonal antibodies raised against one or more drug substances, more particularly immunogenic hapten carrier conjugates formed with said drug substances. The resultant monoclonal anti-drug antibodies can be labeled, or they can be used unlabeled with the labeled or unlabeled drug substances to which they bind in standard testing protocols that have been developed to detect and/or quantify specific binding events with test compounds for predicting biological activity of specifically bound compounds.

In one application of this invention, anti-drug antibodies are substituted for endogenous target molecules for specific binding assays using, for example, capillary electrophoresis (Cetek Corporation, Marlborough, Mass.; U.S. Pat. No. 5,783,397) or surface plasma resonance (Biacore AB, Uppsala, SE; U.S. Pat. No. 5,965,456).

In accordance with another embodiment of the invention the present drug discovery methodology and the identification of useful drug-binding substrates, can be carried out using anti-drug antibodies and drug substances without use of detectable labels. Thus, for example, supernatant test solutions comprising a test compound and a drug substrate in a well of a titer plate containing an immobilized monoclonal antibody raised against the drug substance can be analyzed qualitatively and quantitatively for specific binding events by mass spectral analysis of the supernatant or by chromatographic separation of an aliquot of the supernatant. The relative concentration of the drug substance in the supernatant can be estimated by comparison with concentration calibration curves prepared for each drug substance. The method enables high throughput screening without the cost and time investment required for preparation of labeled antibody and/or drug substance as a component.

In one other method embodiment of the present invention a set of exogenous test compounds are screened for potential biological activity in a living species by contacting a test solution comprising said test compounds or a subset thereof with a drug-binding substrate exhibiting specific affinity for a drug substance having a biological activity known to derive from its specific binding to one or more related endogenous molecules in said species. This method can be carried out by immobilizing the drug-binding substrate or substrates, for example, anti-drug monoclonal antibodies or drug molecule imprinted polymer substrates, in an affinity chromatography column and contacting the immobilized drug-binding substrate with the test solution. The test solution is separated from a drug-binding substrate, and test compounds specifically bound to the substrate are then separated from the substrate and from each other, for example, using liquid chromatography, and then characterized/identified by evaluation of their physical-chemical properties and other structure elucidation techniques.

### DETAILED DESCRIPTION OF THE INVENTION

Commercial drugs (drug substances, herein) typically have unique ADME properties compared to the endogenous ligands they agonize or antagonize. Most therapeutically useful compounds are thus not endogenous ligands that bind to specific receptors or other endogenous biologically functional molecules, rather most useful therapeutics are compounds that not only specifically bind to an endogenous molecule, but they also have favorable absorption, distribution, metabolism, and other bio-properties.

Commercial drug substances have favorable adsorption, distribution, metabolism, and elimination (ADME) properties that endogenous biologically functional ligands frequently lack. Thus endogenous compounds typically cannot be administered effectively as drugs. Dopamine is a classical example. Dopamine is synthesized locally in the central nervous system. Accordingly, the bio-distribution properties needed for endogenous dopamine to act are different than the bio-properties of drugs that must be orally absorbed and penetrate the blood brain barrier for interaction with dopamine receptors. Consequently, dopamine is not a therapeutically useful compounds, i.e., drug, but compounds that bind to the dopamine receptor. Compounds that ultimately become successful commercial drugs must have favorable ADME properties. Thus, dopamine binding substrates are less likely to select test compounds with favorable ADME properties than, for example, halopuridol-binding substrates.

The present invention is directed to a method and related embodiments for screening test compounds for potential biological activity using non-endogenous drug-binding substrates characterized by their specific binding affinity to drug substances. To select for test compounds having potential biological activity, anti-ligand antibodies or anti-drug antibodies, for example, may be used. However, it is hypothesized in accordance with this invention that anti-drug antibodies or other drug-binding substrates formed or identified with known drug substances will enable identification of test compounds with more favorable ADME properties than those test compounds identified by their specific binding to anti-ligand antibodies.

Anti-drug antibodies have been used routinely for both drug-assays and models for studying receptor binding, and receptors and other endogenous biofunctional macromolecules have been used to screen test compounds for biological activity. However, neither anti-drug antibodies nor other non-endogenous drug-binding substrates (those formed or identified using drug substances) have been used as reagents for screening chemical libraries, or screening natural products to identify compounds having potential biological activity corresponding to that of the drug substance used to form or identify the drug-binding substrate. Anti-drug antibodies have a binding site that mimics the topology of the drug(s) against which they are raised, drug(s) which has/have the ADME properties in addition to the ability to bind to a target molecule, necessary for drug efficacy. Thus anti-drug antibodies or other drug-binding substrates formed or identified using drug substance will, according to one premise of this invention, not only specifically bind compounds that may have affinity for the drug's target endogenous macromolecule, but also select for compounds having favorable bio-properties.

The distinction between the use of enzyme or receptor reconstitution assays and anti-drug antibodies for screening purposes in accordance with this invention highlights the significance of the present invention. Functional profiling of compounds against a set of receptors provides information about the binding of compounds to a set of receptors. However, it is well established in drug discovery that such in vitro enzyme/receptor binding assays do not select for compounds that penetrate into cells or that have the necessary distribution properties to become commercial drugs.

The drug discovery selection process has several steps that typically follow a sequence:

Step 1: identify a set of compounds that elicit activity in cell free receptor binding assays;

Step 2: find the compounds in the set with activity in whole cell assays;

Step 3: evaluate the activity in several in vitro screening assays to identify toxicity, cell penetration properties, metabolism, etc.;

Step 4: evaluate the in vivo activity of the compounds in many animal species,

Step 5: identify the compound with activity in humans.

This routine drug discovery process is in general a sequential process and is used to identify compounds that go into Phase I and Phase II clinical trials. A compound that binds to a receptor and has favorable bio-properties will have a higher probability of success in the drug development process than one selected only for its receptor biding activity (Step 1 above).

Accordingly, one working hypothesis of the present invention is that anti-drug antibody binding sites and binding sites on other drug-binding substrates formed or identified with drug substances will specifically bind to compounds that are more likely to have favorable bio-properties and concomitantly a higher probability of becoming a new commercial drug candidate.

Thus, in its broadest perspective, the present method provides a test procedure for identifying test compounds that potentially exhibit a pre-determined biological activity. In one aspect, the method comprises the steps of contacting a solution comprising one or more test compound(s) of unknown or undetermined biological activity, with a drug-binding substrate formed or identified using a drug substance known to exhibit a pre-determined biological activity. The test compound(s) identified as potentially having the pre-determined biological activity are those that exhibit specific binding affinity with the drug-binding substrate. In one embodiment the drug-binding substrate is selected based on its capacity to exhibit specific binding affinity, not only to the drug substance used to form or identify the drug-binding substrate, but as well to a second drug substance known to have the pre-determined biological activity. It is the cross-reactive specific binding affinity of the drug-binding substrates used in accordance with this invention that enables the selection/identification of test compounds potentially having the pre-determined biological activity of the drug substance used to form or identify the drug-binding substrate. Thus the drug-binding substrates preferred for use in accordance with this invention, in contrast to those typically used in classical specific binding ligand/analyte assays, are selected for their cross-reactivity with other drug substances having the pre-determined biological activity.

In another aspect of the invention the test compounds can be drugs of known biological activity which are screened against drug-binding substrates formed or identified with a drug substance having a different biological activity whereby the test drugs are assayed for potential toxicity.

The method of the invention may be used to screen for potential biological activity using well known immunoassay procedures, using polyclonal anti-drug antibodies or fragments thereof, monoclonal anti-drug antibodies or fragments thereof, biosynthetic anti-drug antibody binding sites or other drug-binding proteins such as those presented in phage display libraries. Alternatively the method can be carried out using drug molecule imprinted polymer substrates such as the molecularly imprinted polymer substrate described in U.S. Pat. Nos. 5,872,198; 5,959,050; and 5,994,110. Various specific assay protocols are known per se. See for example, U.S. Pat. Nos. 4,313,734; 4,366,241; 5,783,397; and 5,965,456.

An “endogenous molecule” or “endogenous compound” for the purpose of defining this invention refers to a naturally occurring molecule/compound, exclusive of antibodies, typically genomically encoded or produced by natural biochemical processes, and having biological function relating to mediation of intracellular or intercellular biochemical processes. The term includes, but is not limited to, enzymes, receptors, ion channels, carrier molecules, nucleic acids, chemical derivatives or analogs thereof, and metabolites of exogenous compounds, but it specifically excludes antibodies.

An “exogenous molecule” or “exogenous compound” relevant to a given living species is a molecule or compound having origin external to the species and includes members of combinatorial chemical libraries, other synthetic compounds, and natural products, including extracts comprising endogenous molecules from other species.

A “drug substance” is an exogenous compound that is capable of exhibiting biological activity as a systemic effect by specific binding to one or more structurally related endogenous macromolecules in the species to which it is administered or applied. The term contemplates effective therapeutics for human or animal use and, for example, growth regulators and pesticides, including herbicides and insecticides, for agricultural use.

A “drug-binding substrate” for the purpose of defining the present invention is a chemical entity other than an endogenous molecule that specifically binds to a drug substance. The term includes antibodies raised against drug substances (or, more precisely, against their hapten-carrier conjugates), antibodies raised against other exogenous compounds that specifically bind to said anti-drug antibodies, phage display libraries and molecularly imprinted polymers that bind specifically to drug substances. Drug-binding substrates of biological origin are typically immobilized on a solid carrier for use in this invention.

A “biologically functional molecule” is an endogenous macromolecule known to mediate one or more biochemical processes in a plant, animal or single cell species.

Drug substances having the “same or similar biological activity” are exogenous compounds that 1) specifically bind to one or more structurally related biologically functional molecules, and 2) can be administered or applied to produce a common or related biological effect. Examples of drug substances having the same or similar biological activity are listed for four drug substance categories below:

| Tricyclics | Benzodiazepines |
|---|---|
| Amiltriptyline | Diazepam |
| Doxepin | Clobazam |
| Cyclobenzaprine | Temazepam |
| Nortriptyline | Alprazolam |
| Clomipramine | Nordiazepam |
| Imipramine | Flunitrazepam |
| Trimipramine | Oxazepam |
| Protiptyline | Lometazepam |
| Desipramine | Demoxepam |
| Perphanazine | Clorazepale |
| Chlorpromazine | |
| Prochlorperazine | |
| Triflupromazine | |
| Barbituates | Opiates |
| Bultalbital | Ethylmorphine |
| Aprobarbital | Codeine |
| Secobarbital | Hydrocodone |
| Pentobarbital | Morphine |
| Butabarbital | Thabaine |
| Amobarbital | Hydromorphone |
| Barbital | Levorphanol |
| | Oxycodone |
| | Norcodeine |

A “control compound” as used herein is a drug substance or another non-endogenous compound which specifically binds to an antibody raised against a drug substance.

“Specific binding” or “specific binding affinity” is the affinity of a pair of molecules or a molecule and a chemical structure on a substrate surface deriving from a plurality of specific non-covalent interactions that depend on the three-dimensional structures of the binding entities involved. Typical pairs of specific binding entities include antigen-antibody, hapten-antibody, hormone-receptor, nucleic acid strand-complementary nucleic acid strand, substrate-enzyme, inhibitor-enzyme, carbohydrate-lectin, biotin-avidin, virus-cellular receptor, and the interaction of certain molecules with molecularly imprinted polymer surfaces.

The strength or affinity of binding interactions can be expressed in terms of the dissociation constant (K_{d}) of the interaction, wherein a smaller K_{d} represents a greater affinity. Specific binding properties of various non-endogenous substrates for use in this invention can be qualified and quantified using methods well known in the art. One such method entails measuring the rates of drug substance-recognition substrate complex formation and dissociation, wherein those rates depend on the concentrations of the complexing entities, the affinity of the interaction, and on parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity and is thus equal to the dissociation constant K_{d}. See, generally, Davies et al. (1990) Ann. Rev. Biochem., 59: 439-473.

The phrase “specifically binds to a drug substance or “exhibits specific binding affinity to a drug substance,” when referring to an antibody refers to a binding reaction which is determinative of the presence of the drug substance in the presence of a heterogeneous population of other compounds. Thus, under designated immunoassay conditions, the specified antibodies or other drug-binding substrates useful in carrying out the present invention bind to a particular drug substance and do not bind in a significant amount to other non-cross reacting compounds in the sample. A variety of immunoassay formats may be used to select antibodies specifically binding with one or more drug substance. For example, solid-phase ELISA immunoassays can be used to select monoclonal antibodies specifically immunoreactive with a drug substance or to a drug-carrier conjugate. See Harlow and Lane (1988) Antibodies, A Laboratory Manual, Cold Spring Harbor Publications, New York, for a description of immunoassay formats and conditions that can be used to determine specific immunoreactivity.

The term “antibody” includes both intact antibody molecules of the appropriate specificity and antibody fragments (including Fab, F(ab′), and F(ab′)₂ fragments) as well as chemically modified intact antibody molecules and antibody fragments, including hybrid antibodies assembled by in vitro reassociation of subunits. The term “antibodies” as used herein also refers to all types of immunoglobulins, including IgG, IgM, IgA, IgD, and IgE. An antibody is typically a protein consisting of one or more polypeptide substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “antibody” includes monoclonal antibodies and antibody compositions with polyepitopic specificity (i.e., polyclonal antibodies). The polyclonal antibodies are preferably “affinity purified” antibodies. The term “affinity purified” means that the antibodies have been purified using the antigen or the hapten-carrier conjugate to selectively purify the polyclonal antibodies. Affinity purification can be achieved by immobilizing the antigen on an affinity column (e.g., an agarose column) and passing the polyclonal antibodies through the column. The affinity purified antibodies can be subsequently eluted from the column by changing the elution conditions or by adding a chaotropic agent, for example. For a review of affinity purification techniques with respect to antibodies, see Current Protocols in Immunology, Ed. Coligen et al., Wiley publishers, Vols. 1 and 2, for example.

The antibodies may be of any species of origin, including (for example) mouse, rat, rabbit, horse, or human, or may be chimeric antibodies, and include antibody fragments such as, for example, Fab, F(ab′)₂, and Fv fragments, and the corresponding fragments obtained from antibodies other than IgG. An antibody has specific binding affinity for a ligand or is specific for a ligand if the antibody binds or is capable of binding the ligand as measured or determined by standard antibody-antigen or ligand-receptor assays, for example, competitive assays, saturation assays, or standard immunoassays such as ELISA or RIA. This definition of specificity applies to single heavy and/or light chains, CDRS, fusion proteins or fragments of heavy and/or light chains, that are specific for the ligand if they bind the ligand alone or in combination.

The term “monoclonal antibody” as used herein refers to an antibody obtained from a population of substantially homogeneous antibodies; i.e., the individual antibodies comprising the population are identical except for possible naturally occurring mutations that may be present in minor amounts. Monoclonal antibodies are typically highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The monoclonal antibodies for use in this invention include hybrid and recombinant antibodies produced by splicing a variable (including hypervariable) domain of a selected antibody with a constant domain (e.g.. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity. [See, e.g., U.S. Pat. No. 4,816,567 and Mage & Lamoyi, in Monoclonal Antibody Production Techniques and Applications, pp. 79-97 (Marcel Dekker, Inc., New York (1987)].

Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler & Milstein, Nature, 256: 495 (1975), or may be made by recombinant DNA methods (U.S. Pat. No. 4,816,567). The “monoclonal antibodies” can also be isolated from phage antibody libraries using the techniques described in Clackson et al., Nature, 352: 624-628 (1991) and Marks et al., J. Mol. Biol., 222: 581-597 (1991), for example.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild chemical conditions to cleave the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, NY (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv) antibodies in which a variable heavy and a variable light chain are joined together (directly or through a peptide linker) to form a continuous polypeptide.

In addition to commercially available anti-drug antibodies, one of skill can easily make antibodies specific for drug substances using existing techniques, or modify those antibodies which are commercially or publicly available. In addition to the art referenced above, general methods of producing polyclonal and monoclonal antibodies are known to those of skill in the art. See, e.g., Paul (ed) (1993) Fundamental Immunology, Third Edition Raven Press, Ltd., New York Coligan (1991) Current Protocols in Immunology Wiley/Greene, NY; Harlow and Lane (1989) Antibodies: A Laboratory Manual Cold Spring Harbor Press, NY; Stites et at. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497. Other suitable techniques for antibody preparation include selection of libraries of recombinant antibodies in phage or similar vectors. See, Huse et al. (1989) Science 246: 1275-1281; and Ward et al. (1989) Nature 341: 544-546. Specific monoclonal and polyclonal antibodies and antisera will usually bind with a K_{D} of at least about 0.1 μM, preferably at least about 0.01 μM or better, and most typically and preferably, 0.001 μM or better.

Monoclonal antibodies may also be readily generated using conventional techniques (see U.S. Pat. Nos. RE 32,011; 4,902,614; 4,543,439; and 4,411,993 which are incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference).

Other techniques may also be utilized to construct monoclonal antibodies (see William D. Huse et al., “Generation of a Large Combinational Library of the Immunoglobulin Repertoire in Phage Lambda,” Science 246:1275-1281, December 1989; see also L. Sastry et al., “Cloning of the Immunological Repertoire in Escherichia coli for Generation of Monoclonal Catalytic Antibodies: Construction of a Heavy Chain Variable Region-Specific cDNA Library,” Proc Natl. Acad. Sci USA 86:5728-5732, August 1989; see also Michelle Alting-Mees et al., “Monoclonal Antibody Expression Libraries: A Rapid Alternative to Hybridomas,” Strategies in Molecular Biology 3:1-9, January 1990; these references describe a commercial system available from Stratacyte, La Jolla, Calif., which enables the production of antibodies through recombinant techniques). Briefly, mRNA is isolated from a B cell population, and utilized to create heavy and light chain immunoglobulin cDNA expression libraries in the .lambda.ImmunoZap(H) and .lambda.ImmunoZap(L) vectors. These vectors may be screened individually or co-expressed to form Fab fragments or antibodies (see Huse et al. supra; see also Sastry et al., supra). Positive plaques may subsequently be converted to a non-lytic plasmid which allows high level expression of monoclonal antibody fragments from E. coli.

Similarly, drug-binding substrates for use in this invention may also be constructed utilizing recombinant DNA techniques to incorporate the variable regions of a gene which encode a specifically binding antibody. Within one embodiment, the genes which encode the variable region from a hybridoma producing a monoclonal antibody of interest are amplified using nucleotide primers for the variable region. These primers may be synthesized by one of ordinary skill in the art, or may be purchased from commercially available sources. Stratacyte (La Jolla, Calif.) sells primers for mouse and human variable regions including, among others, primers for V_{Ha}, V_{Hb}, V_{Hc}, V_{Hd}, C_{H}1, V_{L} and C_{L} regions. These primers may be utilized to amplify heavy or light chain variable regions, which may then be inserted into vectors such as ImmunoZAP.™ H or ImmunoZAP.™ L (Stratacyte), respectively. These vectors may then be introduced into E. coli for expression. Utilizing these techniques, large amounts of a single-chain protein containing a fusion of the VH and VL domains may be produced (See Bird et al., Science 242:423-426, 1988). In addition, such techniques may be utilized to change a “murine” antibody to a “human” antibody, without altering the binding specificity of the antibody.

Once suitable antibodies or other drug-binding substrates have been obtained, they may be isolated or purified by many techniques well known to those of ordinary skill in the art (see Antibodies: A Laboratory Manual, Harlow and Lane (eds.), Cold Spring Harbor Laboratory Press, 1988). Suitable techniques include peptide or protein affinity columns, HPLC or RP-HPLC, purification on protein A or protein G columns, or any combination of these techniques.

The word “label” when used herein refers to a detectable compound or composition which can be conjugated directly or indirectly with a molecule such as an antibody or a drug-substance. The label may be detectable by itself (e.g., radioisotope labels or fluorescent labels) or, in the case of an enzymatic label, may catalyze a chemical alteration of a substrate compound or composition which is detectable. Alternatively, a phosphatase enzyme can be used as a label. An example is a chemiluminescent enzyme-linked immunoassay, such as the so-called enzyme-linked immunosorbent assay or ELISA. Such assays, which contain calorimetric or radiometric indicators, are commonly used in manual format as well as on automated multi-test immunoassay systems.

The detectable labels that can be used in carrying out certain embodiments of the method of the present invention, which are attached to either the antibody or the drug substance, can be primary labels (where the label comprises an element that is detected directly or that produces a directly detectable element) or secondary labels (where the detected label binds to a primary label, e.g., as is common in immunological labeling). An introduction to labels, labeling procedures and detection of labels is found in Polak and Van Noorden (1997) Introduction to Immunocytochemistry, 2nd ed., Springer Verlag, NY and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements. Useful primary and secondary labels can include spectral labels such as fluorescent dyes (e.g., fluorescein and derivatives such as fluorescein isothiocyanate (FITC) and Oregon Green.™, rhodamine and derivatives (e.g., Texas red, tetrarhodimine isothiocynate (TRITC), etc.), digoxigenin, biotin, phycoerythrin, AMCA, CyDyes.™, and the like), radiolabels (e.g., ³H, ¹²⁵I, ³⁵S, ¹⁴C, ³²P, ³³P, etc.), enzymes (e.g., horse radish peroxidase, alkaline phosphatase, etc.), spectral colorimetric labels such as colloidal gold or colored glass or plastic (e.g., polystyrene, polypropylene, latex, etc.) beads. The label may be coupled directly or indirectly to a component of the specific binding assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions.

Preferred labels include those that use: 1) chemiluminescence (using horseradish peroxidase and/or alkaline phosphatase with substrates that produce photons as breakdown products as described above) with kits being available, e.g., from Molecular Probes, Amersham, Boehringer-Mannheim, and Life Technologies/Gibco BRL; 2) color production (using both horseradish peroxidase and/or alkaline phosphatase with substrates that produce a colored precipitate [kits available from Life Technologies/Gibco BRL, and Boehringer-Mannheim]); 3) hemifluorescence using, e.g., alkaline phosphatase and the substrate AttoPhos [Amersham] or other substrates that produce fluorescent products; 4) fluorescence (e.g., using Cy-5 [Amersham]), fluorescein, and other fluorescent tags]; 5) radioactivity. Other methods for labeling and detection will be readily apparent to one skilled in the art.

Enzymes can also be conjugated to reagents used to detect and analyze binding events in carrying out the method of the invention include, e.g., beta-galactosidase, luciferase, horse radish peroxidase, and alkaline phosphatase. The chemiluminescent substrate for luciferase is luciferin. One embodiment of a chemiluminescent substrate for .beta.-galactosidase is 4-methylumbelliferyl-beta-D-galactoside. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′adamantane], which is detected with a luminometer. Embodiments of horse radish peroxidase substrates include 2,2′azino-bis(3-ethylbenzthiazoline-6 sulfonic acid)(ABTS), 5-aminosalicylic acid (5AS), o-dianisidine, and o-phenylenediarnine (OPD), which are detected with a spectrophotometer; and 3,3,5,5′-tetramethylbenzidine (TMB), 3,3′diarninobenzidine (DAB), 3-amino-9-ethylcarbazole (AEC), and 4-chloro-1-naphthol (4C1N), which are detected visually. Other suitable substrates are known to those skilled in the art. The enzyme-substrate reaction and product detection are performed according to standard procedures known to those skilled in the art and kits for performing enzyme immunoassays are available as described above.

In general, a detector which monitors a particular probe or probe combination is used to detect the label. Typical detectors include spectrophotometers, phototubes and photodiodes, microscopes, scintillation counters, cameras, film and the like, as well as combinations thereof. Some embodiments of this invention can be implemented using a mass spectrometer optionally in combination with a liquid chromatograph. Examples of suitable detectors are widely available from a variety of commercial sources known to persons of skill. Commonly, an optical image of a substrate comprising bound labeling moieties is digitized for subsequent computer analysis.

The method of the present invention can be carried out using standard immunoassay techniques. In a typical immunoassay, the analyte hapten, antigen or antibody is assayed by detecting the presence or amount of an enzyme-labeled specific binding partner for the analyte or an enzyme label-analyte conjugate. Various assay formats and the protocols for performing the immunochemical steps are well known in the art. These assays fall broadly into two categories. Competitive assays feature an immunological binding of a specific antibody with the analyte and an analyte analog, e.g., a detectably labeled analyte molecule. Sandwich assays result by the sequential or simultaneous binding of two antibodies, one of which is detectably labeled, with the analyte. The detectably labeled binding pair so formed can be assayed with the compounds and methods of the present invention. When the detectable label is the enzyme, it is detected directly. When the detectable label is a member of another specific binding pair, e.g., a hapten, a conjugate of its binding partner with an enzyme is reacted first and the enzyme then detected in accordance with the present methods. Measurement can be performed with enzyme-labeled species attached to a solid surface or support including beads, tubes, microwells, magnetic particles, test strips, membranes and filters such as are in common use in the art. The detectable enzyme-labeled species can also be present free in solution or enclosed within an organized assembly such as a liposome in which case a lytic agent is employed to lyse the liposome and free the detectable enzyme.

The antibodies are generally immobilized on a solid support having surface(s) which are amenable to attachment of such an antibody. By “solid support” is meant a non-aqueous matrix drug-binding substrate can adhere. The solid phase can comprise a discontinuous solid phase of discrete particles. The particles can be porous and formed from a number of different materials, e.g., polysaccharides (e.g., agarose), polyacrylamides, polystyrene, polyvinyl alcohol, silicones and glasses. For examples of suitable particulate solid phases, see U.S. Pat. No. 4,275,149. Other suitable examples include without limitation microtiter plates; membrane material such as nitrocellulose, cellulose, cellulose acetate, polycarbonate, etc.; test tubes; particles such as beads, column packing materials, etc.; and derivatized surfaces having binding domains capable of binding the antibody attached thereon. In a preferred embodiment the antibody is a coating antibody which is fixed to a solid support.

Antibody assays (immunoassays) may, in general, be homogeneous assays or heterogeneous assays. In a typical homogeneous assay the immunological reaction usually involves the specific antibody, a labeled analyte, and the set of test compounds. In some assays the antibody itself is labeled and in others the analyte (drug substance) is labeled, depending on the methodology employed for detecting specific binding events. The signal arising from the label is modified, directly or indirectly, upon the binding of the antibody to the labeled analyte. Both the immunological reaction and detection of the extent thereof are typically carried out in a homogeneous solution. Immunochemical labels which may be employed include free radicals, radioisotopes, fluorescent dyes, enzymes, bacteriophages, coenzymes, and so forth.

In a heterogeneous assay approach, the reagents are usually the test compound or the set of test compounds, one or more anti-drug antibodies and a system or means for producing a detectable signal. The antibody can be immobilized on a solid support, such as a bead, plate or slide, and contacted with the set of test compounds in a liquid phase. The support is then separated from the liquid phase and either the support phase or the liquid phase is examined for a detectable signal employing means for producing such signal. The location of the signal is related to the binding of one or more test compounds to the antibody. Means for producing a detectable signal include the use of radioactive labels, fluorescent labels, enzymes for use in colorimetric assays, and so forth. The extent of retention of the detectable group on the solid support indicates the presence or absence of a competitively binding test compound in the test sample. Examples of suitable immunoassays are the radioimmunoassay, immunofluorescence assays, enzyme-linked immuno adsorbant assays, and the like.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al., U.S. Pat. No. 4,376,110 to David et al., U.S. Pat. No. 4,275,149 to Litman et al., U.S. Pat. No. 4,233,402 to Maggio et al., and U.S. Pat. No. 4,230,767 to Boguslaski et al.

In accordance with the present invention a drug-binding substrate formed or identified using a drug substance having a predetermined biological activity is used to screen or identify test compounds likely to exhibit the predetermined biological activity. The potential biologically active test compounds are identified by their specific binding to the drug-binding substrate(s) as detected by any of a wide variety of specific binding assay techniques using labeled or unlabeled assay components. In one embodiments a monoclonal antibody raised against a drug substance is used as a drug-binding substrate to identify and isolate test compounds in a natural product extract or other source of test compounds, for example, a chemical library for natural or synthetic sources. Preferably, the monoclonal antibody exhibits specific binding to at least one other drug substance having the same or a similar biological activity as the drug substance against which the antibody was raised. The invention finds use inter alia in drug discovery protocols, in toxicity profiling of drug substances, and in assaying commercial natural products.

In accordance with one embodiment of the present invention there is provided a method of identifying possible drug substances or lead compounds for drug substances having a predetermined biological activity in a living species, including single cell organisms or multicellular organisms including, for example, plants and animals. The method comprises the steps of contacting a test solution comprising an exogenous test compound with a drug-binding substrate capable of specific binding to a drug substance known to exhibit the predetermined biological activity in the living species. The test compounds having potential for exhibiting the predetermined biological activity are identified by detecting the existence of specific binding of the test compound to the drug-binding substrate. In one embodiment the method further comprises the step of determining whether or not the test compounds that specifically bind to the drug-binding substrate exhibit the biological activity. That determination can be made by independent testing in known biological assays or animal tests designed to detect or confirm the predetermined biological activity.

The “drug-binding substrate” for use in accordance with the present invention is formed or identified using one or more drug substances having a predetermined biological activity. Thus, in one embodiment the drug-binding substrate is an antibody, more preferably a monoclonal antibody, raised against a drug substance having the predetermined biological activity. Alternatively the drug-binding substrate can be a drug molecule imprinted polymer substrate. The preparation of molecularly imprinted polymer substrates is known in the art. See, for example, U.S. Pat. Nos. 5,872,198; 5,959,050; and 5,994,110. Alternatively, the drug-binding substrates can be identified empirically by exposing sources of substrates of high diversity, for example, those available as phage display libraries, to labeled drug substances and identifying those substrates that exhibit specific binding affinity to the drug substance.

Preferably, “drug-binding substrates” for use in the drug discovery testing embodiments of the invention are selected based on their cross-reactivity with at least one other drug substance having the same biological activity as the drug substance used to form or identify the drug-binding substrate. Thus, in accordance with one embodiment of the present invention there is provided a method for defining a drug-binding substrate for use in drug discovery testing. The method comprises the step of contacting potential drug-binding substrates with a solution of a drug substance having a known biological activity deriving from its specific binding to one or more related endogenous molecules. The substrates exhibiting specific binding to the drug substance are identified and thereafter at least those substrates are contacted with a solution of a second drug substance exhibiting the predetermined biological activity.

One preferred class of “drug-binding substrates” for use in the present invention are those substrates that exhibit specific binding to each of the first and second drug substances. Alternatively, “drug-binding substrates” for use in the present invention can be identified by contacting potential drug-binding substrates with a solution comprising a plurality of drug substances, each having a common biological activity deriving from specific binding to one or more related endogenous molecules. Those substrates identified for their specific binding to one or more of the drug substances can be employed for use in the screening/analysis aspects of the present invention. Optionally the potential “drug-binding substrates” can be contacted with a solution of a third drug substance exhibiting the same or similar biological activity as the first and second drug substances used to identify the drug-binding substrates, and those substrates that exhibit specific binding to at least two of said drug substances are employed for use in drug screening/analysis aspects of the invention. One procedure for implementing the method for defining the drug-binding substrates for use in the present invention is to contact a plurality of potential drug-binding substrates with a solution comprising a known concentration of a drug substance, for example, each of a series of potential drug-binding substrates can be located/immobilized in a well of a microtiter plate and contacted with a solution comprising a known concentration of a drug substance. The concentration of the drug substance in the solution after contact with the potential drug-binding substrate can be determined, for example, by mass spectrographic analysis of a volume of the test solution. Mass peak intensities correspond to the relative concentration of the drug substance in the supernatant test solution. The drug-binding substrates located in those wells where the supernatants are determined to have a reduced concentration of the drug substance are indicated for specific binding to that drug substance. Other methods of analyzing drug substance concentration in the supernatant can be employed, for example, quantitative chromatographic analysis. Alternatively, labeled analogs of the drug substance can be used to detect specific binding using standard specific binding assay protocols.

In a related embodiment of the present invention there is provided a composition for analysis or screening of compounds for biological activity. The composition comprises at least first and second drug-binding substrates wherein the first substrate exhibits specific binding to a first drug substance having a known biological activity deriving from its specific binding to one or more related endogenous molecules and the second drug bonding substrate exhibits specific binding to the same drug substance or to another drug substance having the same or similar biological activity to the first drug substance. In one embodiment of that composition, at least one of the first or second drug binding substrates exhibits specific binding to another drug substance other than the first or second drug substance but one having the same or similar biological activity as the first and second drug substances. The drug-binding substrates can be antibodies, most typically monoclonal antibodies, which are optionally immobilized on a solid support, ready for use in the analysis or screening embodiments of this invention. The drug-binding substrates formed or identified using drugs of known biological activity can also be used as components of a kit for analysis or screening of test compounds for potential biological activity. In one embodiment the kit comprises at least first and second monoclonal antibodies. The first monoclonal antibody is raised against a first drug substance having a predetermined biological activity, and the second monoclonal antibody is raised against either the first drug substance or a second drug substance having the same or similar biological activity. In one embodiment the monoclonal antibody components exhibit specific binding to another drug substance exhibiting the same biological activity, but a drug substance other than the drug substance against which each of said monoclonal antibodies was raised. In a related kit embodiment for analysis or screening of test compounds for potential biological activity, the kit comprises at least first and second monoclonal antibodies wherein the first monoclonal antibody is raised against a first drug substance having a predetermined biological activity and the second monoclonal antibody is raised against another monoclonal antibody having the same or a different biological activity. In that embodiment the kit can be used to screen test compounds for more than one potential biological activity in a parallel processing protocol. Preferably, each of said first or second antibodies exhibit specific binding to another drug substance having the same biological activity as the drug substance against which the respective antibodies were raised. The antibodies are preferably presented in the kit already immobilized on a solid substrate for use in screening test compounds for biological activity, for qualitative analysis of natural products, or for toxicity profiling of biologically active compounds in accordance with those respective embodiments of this invention.

In one such embodiment of the invention there is provided a method for screening a test compound or a set of test compounds for potential biological activity. The test compounds can be components of a natural product comprising an extract of tissue or plant, fungal, microbial or marine origin, members of a combinatorial chemical library, or one or more compounds that have a biological activity different than that for which the compounds are being screened. The method comprises the steps of selecting a drug-binding substrate exhibiting specific binding to a drug substance having the biological activity for which the test compounds are being screened, a biological activity typically derived from specific binding to one or more related endogenous molecules, and contacting the drug-binding substrate with a test solution comprising (1) a test compound and (2) a known amount of the drug substance or a second drug substance known to bind to said substrate competitively with said drug substance, for a period of time sufficient to allow specific binding of the drug substance to the drug-binding substrate. Finally, the supernatant test solution in contact with the drug-binding substrate is analyzed to determine the extent, if any, to which a test compound blocks binding of the substrate with the first or second drug substance. This can be accomplished, for example, by measuring the amount of the drug substance in the test solution or the amount of drug substance bound to the substrate in contact with the test solution. That measurement can be carried out using any one or more of several analytical techniques, for example, a known volume of the supernatant can be subjected to chromatographic separation and analysis after the period of time for specific binding. A quantitative analysis can be achieved by comparing the chromatogram, for example, from a high pressure liquid chromatograph of the aliquot from the supernatant with a preestablished quantitative calibration curve for the drug substance. That method of analysis is particularly well adapted for screening procedures wherein the test solution comprises multiple test compounds, for example, as would be the case in assaying natural product extracts or multiple members of a combinatorial library. Where the test solution contained a single test compound in combination with the known amount of a drug substance, the extent to which that test compound blocks binding of the drug-binding substrate with the drug substance can be assayed simply by mass spectrographic analysis of a fraction of the test solution after the period of time for specific binding. The relative amount of the drug substance in the test solution can be determined simply by comparing mass peak intensities obtained with a known volume of the test solution with a pre-established quantitative calibration curve for the drug substance in the mass spectrograph. Alternatively, the method can be carried out with a labeled drug substance and the extent that the test compound blocks binding of the substrate with the drug substance can be determined using art-recognized label-dependent procedures.

In a related screening method embodiment of the invention, a set of exogenous test compounds is screened for potential biological activity using a monoclonal antibody exhibiting specific binding for a control compound known to exhibit specific binding to a biologically functional endogenous molecule in a living species. The method comprises the steps of contacting the set of test compounds, typically in solution, with a drug-binding substrate comprising the monoclonal antibody under conditions conducive to specific binding between the antibody and at least one of the test compounds to form an antibody-test compound complex. At least a portion of the complex is separated from at least a portion of the non-complex test compounds, and the complex test compound is then identified using art-recognized procedures including the steps of subjecting the complex to conditions effective to separate the complex test compounds from the monoclonal antibody and thereafter isolating the test compound or test compounds by, for example, chromatographic separation techniques. The potential biological activity of the test compounds can then be further confirmed by measuring the affinity of the test compound forming the antibody-test compound complex for the biologically functional endogenous molecule, such as an enzyme, a receptor, a transport protein, for example an ion transporter or a carrier molecule or a nucleic acid. Alternatively, the test compounds forming antibody-test compound complexes can be subjected to further screening by contacting those compounds with a second antibody exhibiting specific binding to a second control compound known to exhibit specific binding to the same biologically functional endogenous molecule, and identify those test compounds that form antibody-test compound complexes with each antibody.

In a related method embodiment of this invention there is provided a method for screening a set of test compounds for potential biological activity, including the steps of contacting a solution of the set of test compounds or a subset thereof with a substrate comprising an antibody, a phage display library, or a molecularly imprinted polymer exhibiting specific binding to a control compound known to exhibit specific binding to a biologically functional molecule. That contacting step is carried out under conditions conducive to specific binding between the substrate and any test compounds to which it specifically binds to form one or more complexes of the substrate and the respective test compounds. The substrate-test compound complex is separated from at least a portion of the test solution and the test compounds forming complexes with the substrate are identified. In one preferred embodiment of that method the control compound is a drug substance and the substrate comprising a monoclonal antibody raised against that drug substance or a phage display library determined to bind specifically to said drug substance. The complex test compounds can then be separated from the binding substrate and from each other, optionally further assayed by determining specific binding for the biologically functional molecule, and identified by art-recognized structure elucidation techniques. In an alternate embodiment of that screening method the solution of test compounds can be contacted with at least first and second substrates, the first substrate exhibiting specific binding to a first control compound known to exhibit specific binding to the biologically functional molecule and the second substrate exhibiting specific binding either to said first control compound or to a second control compound known to exhibit specific binding to the biologically functional molecule.

The complexes formed between the substrate and a test compound are useful for predicting the biological activity of the test compound. Typically the test compound is not known to exhibit the predicted biological activity or to exhibit specific binding to an endogenous molecule associated with said biological activity. The complex comprises said test compound specifically bound to a substrate known to exhibit specific binding to a drug substance known to exhibit said biological activity. The substrate component of such complexes can be, for example, an antibody, a member of a phage display library, or a molecularly imprinted polymer substrate formed from or identified by a drug substance known to exhibit the biological activity. In one preferred embodiment the substrate is a monoclonal antibody raised against the drug substance and, more preferably, one that exhibits specific binding to at least one other drug substance known to exhibit the biological activity.

In one preferred aspect of the present invention there is provided a method of using a monoclonal antibody for screening compounds for probable biological activity. The method comprises the step of selecting a monoclonal antibody raised against a drug substance exhibiting the biological activity and capable of specific binding to said drug substance and at least one other drug substance known to exhibit said biological activity. The monoclonal antibody is contacted with a solution comprising the test compounds and an antibody-test compound complex is formed, and specific binding of the antibody to a test compound is detected. The test compound forming the antibody-test compound complex is thereafter identified. Preferably the method includes the additional step of separating at least a portion of the antibody-test compound complex from at least a portion of the test compound solution. The monoclonal antibody is preferably immobilized on a solid support, for example, a microtiter plate. Depending on the method used to detect the formation of the antibody-compound complex, the test solution can further include the drug substance used to form the drug substance covalently linked to a detectable label. In instances where the test solution also includes a known concentration of the drug substance, the formation of the complex can be detected by a method comprising mass spectroscopic analysis of an aliquot of a test solution or by a quantitative chromatographic separation/analysis thereof.

Recently numerous new methodologies and instrumentation has been developed for studying and measuring specific binding events between ligands and biologically significant molecules. Thus, for example, assay instruments based on measuring the change in surface optical characteristics of a sensor chip using surface plasmon resonance is available from Biacore AB. Biological functional molecules are immobilized on the sensor surface and changes in surface optical characteristics are measured as a solution of a test compound or compounds is flowed across the sensor surface. Surface plasmon resonance is an optical phenomenon that is used to measure changes in the solution concentration of molecules at a biospecific surface, for example, that of the sensor chip having immobilized biologically functional molecules. The resonance signal depends on the refractive index of solutions in contact with the surface. Molecules and solutions exhibit changes in refractive index and thus give rise to a measurable resonance signal if a biospecific interaction occurs. Typically protein or DNA is immobilized by one of several possible methods onto a carboxy methylated dextran-gold surface. The interacting compound of interest is injected over the surface and the kinetics of binding is measured in real time.

Such instrumentation is described in U.S. Pat. Nos. 5,641,640; 5,955,729; 5,965,456; and 5,972,612. Another recently described methodology for studying specific binding interactions utilizes capillary electrophoresis. Target molecules are combined with test compounds potentially capable of exhibiting specific binding to the target molecule and the mixture is subjected to capillary electrophoresis. Charged compounds present in the sample that bind to the target molecule alter its normal migration time upon capillary electrophoresis, by changing its charge-to-mass ratio, or to effect a variation in peak shape or area. Specific binding events can be detected by simply monitoring the migration of the target molecule during electrophoresis. The method utilizing capillary electrophoresis is described in U.S. Pat. No. 5,783,397.

In one aspect of the present invention, an antibody, preferably a monoclonal antibody, can be substituted for the potential target molecule or biologically functional molecule, the antibody being selected for its specific binding affinity to a drug substance known to exhibit a predetermined biological activity.

The term “toxicity profiling” as used herein with reference to one application of the present invention refers to the use of a panel of anti-drug antibodies or other drug-binding substrates, each capable of specific binding to a unique drug-binding substrate to screen a putative drug substance for cross-reactivity with one or more drug-binding substrates for unrelated drug substances, the existence of which cross-reactivity is suggestive of undesired biological activity and consequent potential toxicity. Thus, the toxicity profiling of a compound identified as a new drug substance against a panel of anti-drug antibodies to a plurality of drug substances having biological activities unrelated to that of the new drug substance should provide useful information predictive of potential side effects deriving from one or more detected secondary biological activities. As an example of implementation of this application of the present invention, each of at least a portion of the wells in a 96-well titer plate is coated with a unique but known anti-drug monoclonal antibody. At least a portion of the anti-drug antibodies are selected for their capacity to bind specifically to at least two drug substances exhibiting the same or similar biological activity. Some wells are left uncoated and others are coated with a monoclonal antibody to the test drug substance. Each of the wells is loaded with an equal volume of a solution of the test drug substance. After an incubation period, adequate to allow specific binding events to reach equilibrium in the respective wells, a volume of the supernatant from each well is injected into a mass spectrometer (previously calibrated with varying amounts of the test drug substance). The relative concentration of the test drug substance in the supernatant of each well provides a measure of the specific binding of the drug substance by the respective anti-drug antibodies. Specific binding of the test drug with antibodies other than the antibody raised from a test-drug hapten conjugate, or other than antibodies raised from immunogenic conjugates of a drug substance having the same or similar biological activity as the test drug substance, is predictive of unwanted biological activity and concomitantly potential toxicity.

In another embodiment of this invention natural products, for example, extracts of plant, microbial, fungal, yeast or marine species are subjected to evaluation for specific binding of certain components to drug-binding substrates, themselves selected for their capacity to bind specifically to drug substances having the same or similar activity as one or more biologically significant components of the natural product. The method can be used to identify the presence of previously unknown biologically active natural products in complex natural product mixtures or it can be used, for example, as a means for enabling quality control testing on commercially available extracts being sold for human or animal use. The method can be implemented using any of a wide variety of testing protocols heretofore developed and described generally hereinabove for detecting specific binding events using either labeled drug substances (corresponding to the anti-drug antibody or other drug-binding substrate) in competitive binding-based assays or labeled anti-drug antibodies for use in capillary zone electrophoresis-based assays (See, e.g., U.S. Pat. No. 5,783,397).

Other competitive or non-competitive binding assay protocols can be employed alone or in combination with chromatography and/or, for example, mass spectral detection/analysis without use of labeled drug substances or labeled antibodies. Thus, unknown, potentially biologically active components of a natural product extract can be separated from the extract and ultimately from each other by first employing affinity chromatography techniques using immobilized anti-drug antibodies and subsequent non-affinity chromatographic separation techniques using mass spectrometry or other suitable detectors allowing identification of the bound components. On the other hand, a natural product mixture detailed for a defined biological activity can be analyzed for such activity by using, for example, a microtiter plate having at least a portion of the wells coated with one or more antibodies, preferably monoclonal antibodies, raised to one or more drug substances having the same or similar biological activity as the component of natural product said to have such activity. A solution of the natural product mixture and a known amount of the drug substance against which the respective antibodies were raised are added to each well. After a period of time sufficient to allow competitive binding to be at or near equilibrium, aliquots from one or more wells are subjected to chromatographic separation, and the relative amounts of each drug substance in the supernatant in the antibody-containing wells are compared with the amounts of the drug substance in the supernatants of both positive control and negative control wells to provide an indication of the presence and the relative amount of the biologically active component or components of the natural product. Estimates of the quantitative values can be obtained by establishing a drug substance concentration calibration curve for the chromatographic separation apparatus, for example a high pressure liquid chromatograph, and comparing the elution profiles of the supernatant aliquots from the wells with the calibrated values. The foregoing procedures can also be applied with little, if any, modification to the screening of test compounds in chemical libraries and other compounds from natural or synthetic sources.

General Procedure for LC/MS Assay for High Throughput Screening

The first step is the development an HPLC assay for separating up to 50 drugs (drug substances) at a time using a reference mass spectrum (MS) library (a MS of each compound), a liquid chromatograph/mass spectrometer (LC/MS) automated peak picking method to find each drug, and a data analysis system to detect each compound.

Quantitative Methods

Step 1: Create a reference mass spectra of each drug (determine which compounds are + Vs. − mode).

Step 2: Create a standard curve for each drug, determine the limits of detection, sensitivity, and any other parameter needed.

Step 3: Create a chromatographic condition that allows all compounds to be separated on a chromatography column.

Assay

Step 1: An anti-drug antibody is located in each well or container.

Step 2: Immobilization method (e.g., ELISA plate).

Step 3: Incubate drug(s) (corresponding to the antibody) and test compound(s) in each well (e.g., ELISA plate with 96 different anti-drug antibodies). The same sample may be applied to each well. This set up will work for individual compounds, and synthetic and natural product libraries.

Step 4: Pool aliquots from each well and quantitate the amount of free drug in solution using the LC/MS library established method.

Knowing the mass of each of the respective drugs for quantitation and where to look for the compounds during the chromatographic separation, one is able to quantitate the compound of drug in the supernatant in each well. If a natural product is screened, the chance for an overlapping molecular weight exactly equal to the elution of the drug is minimal. The amount of the drug in the supernatant is proportional to the amount, if any, of test compound(s) that compete with the drug for the immobilized anti-drug antibody in the well. The need for a reference library is decreased as the resolution of the detector increases. In other words, by using a high resolution mass spectrometer with accurate mass out to 3-4 decimals, need for a reference library is not as great.

1. A method of identifying new drug substances or lead compounds for new drug substances having a predetermined biological activity in a living species, said method comprising the steps of contacting a test solution comprising an exogenous test compound of unknown biological activity with a drug-binding substrate formed or identified using a drug substance known to exhibit the predetermined biological activity in said species and capable of specific binding to said drug substance, detecting the existence of specific binding of the test compound with the drug-binding substrate, and determining whether the specific binding test compound exhibits the predetermined biological activity.

2. The method of claim 1 wherein the drug-binding substrate is an antibody or a phage display library.

3. The method of claim 1 wherein the drug-binding substrate is a monoclonal antibody raised against the drug substance.

4. The method of claim 3 wherein the antibody specifically binds to a second drug substance having the predetermined biological activity.

5. The method of claim 1 wherein the test solution comprises an extract of a natural product.

6. The method of claim 1 wherein the test solution comprises a member of a combinatorial chemical library.

7. The method of claim 5 or claim 6 wherein the drug-binding substrate is a monoclonal antibody raised against the drug substance.

8. A method for screening a test compound or a set of test compounds for potential biological activity, said method comprising the steps of
selecting two drug-binding substrates formed or identified using a drug substance exhibiting said biological activity deriving from specific binding to one or more related endogenous molecules and exhibiting specific binding affinity for said drug substance;
contacting said substrate with a test solution comprising 1) a test compound of unknown biological activity and 2) a known amount of said drug substance, or a second drug substance known to bind to said substrate competitively with said drug substance, for a period of time sufficient to allow specific binding of the drug substance and the drug-binding substrate;
determining whether the test compound blocks binding of the substrate with said first or second drug substance; and
determining whether the test compound that does block said binding exhibits the biological activity.

9. The method of claim 8 wherein the drug-binding substrate comprises an antibody.

10. The method of claim 8 wherein the determination of the extent of blocked binding of the drug substance and the substrate comprises the step of measuring the amount of drug substance in the test solution or the amount of drug substance bound to the substrate.

11. The method of claim 8 wherein the drug-binding substrate exhibits specific binding affinity for at least first and second drug substances having the biological activity.

12. The method of claim 10 wherein the drug-binding substrate exhibits specific binding affinity for at least first and second drug substances having the biological activity.

13. The method of claim 8 wherein measurement of the amount of drug substance is carried out by a method comprising chromatographic separation of the test solution after the period of time for specific binding.

14. The method of claim 8 wherein measurement of the amount of drug substance is carried out by a method comprising mass spectrographic analysis of a fraction of said test solution after the period of time for specific binding.

15. The method of claim 8 wherein the set of test compounds is a natural product comprising an extract of tissue of plant, fungal, microbial or marine origin, or members of a combinatorial chemical library.

16. The method of claim 8 wherein a plurality of test solutions comprising test compounds of unknown biological activity are contacted with a plurality of respective unique drug-binding substrates.

17. A method for detecting potential toxicity of a new drug substance having a first biological activity, said method comprising the steps of
contacting a test solution comprising said new drug substance with a drug-binding substrate formed or identified using a known drug substance known to exhibit a unique second biological activity, said drug-binding substrate capable of specific binding to said known drug substance;
detecting the existence of specific-binding of the new drug substance to the drug-binding substrate; and
determining whether specific-binding new drug substance exhibits the unique second biological activity.

18. The method of claim 17 wherein the drug-binding substrate is an antibody capable of specific binding to the known drug substance.

19. The method of claim 18 wherein the test solution is contacted with a multiplicity of unique substrates each capable of specific binding to known drug substances.

20. A method for identifying a drug-binding substrate for use in drug discovery testing, said method comprising the steps of
contacting potential drug-binding substrates with a solution of a drug substance having a known biological activity deriving from its specific binding to one or more related endogenous molecules;
identifying those substrates to which the drug substance exhibits specific binding affinity;
contacting a solution of a second drug substance exhibiting said known biological activity with at least the substrates exhibiting specific binding affinity to said first drug substance; and
identifying those substrates that exhibit specific binding to each of said first and said second drug substances.

21. A method for identifying a drug-binding substrate for use in drug discovery testing, said method comprising the step of contacting potential drug-binding substrates with a solution comprising a plurality of drug substances, each having a common biological activity deriving from specific binding to one or more related endogenous molecules, and identifying those substrates which specifically bind to more than one of said drug substances.

22. The method of claim 20 or claim 21 wherein the potential drug-binding substrates are antibodies.

23. The method of claim 20 or claim 21 wherein the potential drug-binding substrates are monoclonal antibodies.

24. The method of claim 20 or claim 21 wherein the potential drug-binding substrates are phage display libraries.

25. The method of claim 20 or claim 21 wherein the drug-binding substrates are molecularly imprinted polymers.

26. The method of claim 20 or claim 21 further comprising the step of contacting the potential drug-binding substrates with a solution of a third drug substance exhibiting said known biological activity and identifying those substrates that exhibit specific binding affinity to at least two of said drug substances.

27. The method of claim 20 or claim 21 wherein the step of identifying those substrates that specifically bind to the drug substance comprises the step of analyzing the concentration of the drug substance in the test solution after contact with the substrate said analysis comprising mass spectrographic analysis of a volume of the test solution.

28. A composition for analysis or screening of compounds for potential biological activity, said composition comprising at least first and second drug-binding substrates, said first substrate exhibiting a specific binding affinity to a first drug substance having a known biological activity deriving from its specific binding to one or more related endogenous molecules and said second drug-binding substrate exhibiting specific binding affinity to the same drug substance or to another drug substance having the same or similar biological activity as the first drug substance.

29. The composition of claim 28 wherein the first and second drug-binding substrates exhibit specific binding affinities to another drug substance other than the first or second drug substance but having the same or similar biological activity as the first and second drug substances.

30. The composition of claim 29 wherein the drug-binding substrates are monoclonal antibodies.

31. The composition of claim 30 wherein the monoclonal antibodies are immobilized on solid support.

32. A method for screening a set of exogenous test compounds for potential biological activity, said method comprising the steps of
selecting a monoclonal antibody exhibiting specific binding affinity for a control compound known to exhibit specific affinity for a biologically functional endogenous molecule in a living species;
contacting the set of test compounds with a drug-binding substrate comprising the antibody under conditions conducive to specific binding between the antibody and at least one of the test compounds to form an antibody-test compound complex;
separating at least a portion of the complex from at least a portion of the non-complexed test compounds;
identifying the complexed test compound; and
measuring the affinity of the test compound forming the antibody-test compound complex for the biologically functional endogenous molecule.

33. The method of claim 32 wherein the control compound is a drug substance.

34. The method of claim 32 or claim 33 wherein the selected antibody exhibits specific affinity for at least one other control compound known to exhibit specific affinity for the biologically functional endogenous molecule.

35. The method of claim 32 wherein the complexed test compound is identified by a procedure comprising mass spectroscopy.

36. The method of claim 32 or claim 33 wherein the antibody exhibiting specific affinity for the control compound also exhibits specific affinity for at least two other control compounds known to exhibit affinity for the biologically functional molecule.

37. The method of claim 32 wherein the biologically functional molecule is an enzyme.

38. The method of claim 32 wherein the biologically functional molecule is a transport protein.

39. The method of claim 32 wherein the biologically functional molecule is selected from the group consisting of enzymes, receptors, ion channels, carrier molecules, and nucleic acids.

40. The method of claim 32 or claim 33 further comprising the step of contacting the set of test compounds with a second antibody exhibiting specific affinity for the first and second control compounds and identifying the test compounds that form antibody-test compound complexes with each antibody.

41. The method of claim 32 or claim 33 wherein the set of test compounds comprises member compounds of a combinatorial chemical library.

42. The method of claim 32 or claim 33 wherein the set of test compounds comprises a natural product comprising an extract of a plant, fungus, bacterial or marine species.

43. A method for screening a set of test compounds for potential biological activity, said method comprising the steps of contacting a solution of the set of test compounds or a subset thereof with a substrate comprising an antibody, a phage display library or a molecularly imprinted polymer exhibiting specific binding affinity for a control compound known to exhibit specific affinity for a biologically functional molecule, said contacting step being carried out under conditions conducive to specific binding between the substrate and any test compounds to which it specifically binds to form one or more complexes of the substrate and the respective test compounds, separating the substrate-test compound complex from at least a portion of the solution, identifying a complexed test compound, and measuring the affinity of the complexed test compound for the biologically functional molecule.

44. The method of claim 43 wherein the control compound is a drug substance.

45. The method of claim 43 or claim 44 wherein the substrate comprises a monoclonal antibody or a phage display library.

46. The method of claim 43 or claim 44 wherein the substrate comprises a molecularly imprinted polymer surface formed to exhibit specific binding affinity for the control compound.

47. The method of claim 43 or claim 44 further comprising the step of determining the affinity of the test compound forming a complex with the substrate for the biologically functional molecule.

48. The method of claim 43 or claim 44 wherein the set of test compounds comprises members of a combinatorial library.

49. The method of claim 43 or claim 44 wherein the set of test compounds comprises an extract of tissue of plant, fungal, microbial, or marine origin.

50. The method of claim 43 or claim 44 wherein the substrate exhibits specific affinity for at least one other control compound known to exhibit specific binding affinity for the biologically functional molecule.

51. The method of claim 43 or claim 44 wherein the solution of test compounds is contacted with at least first and second substrates, said first substrate exhibiting specific binding affinity for a first control compound known to exhibit specific binding affinity for the biologically functional molecule and said second substrate exhibiting specific binding affinity either for said first control compound or for a second control compound known to exhibit specific binding affinity for the biologically functional molecule.

52. A complex useful for predicting biological activity of a test compound not known to exhibit said biological activity or to exhibit specific binding to an endogenous molecule associated with said biological activity, said complex comprising said test compound specifically bound to a substrate known to exhibit specific binding affinity for a drug substance known to exhibit said biological activity.

53. The complex of claim 52 wherein the substrate is selected from the group consisting of an antibody, a member of a phage display library, and a molecularly imprinted polymer substrate.

54. The complex of claim 52 wherein the substrate is a monoclonal antibody raised against the drug substance.

55. The complex of claim 53 wherein the substrate exhibits specific binding affinity for at least one other drug substance known to exhibit said biological activity.

56. The complex of claim 54 wherein the substrate exhibits specific binding affinity for at least one other drug substance known to exhibit said biological activity.

57. An apparatus for screening test compounds for biological activity, said apparatus comprising a solid substrate exhibiting specific binding affinity for a control compound having known biological activity;
a vessel for containing a solution comprising a test compound and the control compound, optionally covalently linked to a detectable label, in contact with said substrate;
a detector for sensing the formation of substrate-test compound complexes; and
a chromatographic system for separating the control compound from at least a portion of the test compounds.

58. The apparatus of claim 57 wherein the chromatographic system is HPLC.

59. The apparatus of claim 57 wherein the solid substrate comprises an antibody, a member of a phage display library, or a molecularly imprinted polymer surface.

60. The apparatus of claim 57 wherein the substrate exhibits specific binding affinity for at least one other control compound known to exhibit the biological activity.

61. The apparatus of claim 57 wherein the control compound is a drug substance.

62. The apparatus of claim 61 wherein the substrate comprises a monoclonal antibody.

63. The apparatus of claim 57 wherein the detector comprises a mass spectrometer.

64. The apparatus of claim 57 wherein the detector comprises a fluorescence detector.

65. A kit for analysis or screening of test compounds for potential biological activity, said kit comprising at least first and second monoclonal antibodies, said first monoclonal antibody being raised against a first drug substance having a biological activity, and said second monoclonal antibody being raised against either said first drug substance or a second drug substance having said same biological activity, and wherein said monoclonal antibodies exhibit specific binding to a drug substance other than the drug substances against which each of said monoclonal antibodies was raised.

66. A kit for analysis or screening of test compounds for potential biological activity, said kit comprising at least first and second monoclonal antibodies, said first monoclonal antibody being raised against a first drug substance having a predetermined biological activity and said second monoclonal antibody being raised against another drug substance having the same or a different biological activity.

67. The kit of claim 66 wherein each of said first and second antibodies exhibit specific binding to another drug substance having the same biological activity as the drug substances against which the antibodies were raised.

68. A method of using a monoclonal antibody for screening test compounds of unknown biological activity for a probable biological activity, said method comprising the steps of selecting a monoclonal antibody raised against a drug substance exhibiting said biological activity and capable of specific binding to said drug substance and at least one other drug substance known to exhibit said biological activity,
contacting said monoclonal antibody with a solution comprising said test compounds;
detecting the formation of an antibody-test compound complex formed by specific binding of the antibody to a test compound;
identifying the test compound forming the antibody-test compound complex; and
testing said test compound for said biological activity.

69. The method of claim 68 further comprising the step of separating at least a portion of the antibody-test compound complex from at least a portion of the test compound solution.

70. The method of claim 68 or claim 69 wherein two or more monoclonal antibodies are immobilized on solid substrates.

71. The method of claim 68 or claim 69 wherein the solution further comprises the drug substance covalently linked to a detectable label.

72. The method of claim 68 or claim 69 wherein the solution further comprises the drug substance.

73. The method of claim 68 or claim 69 wherein the formation of the complex is detected by a method comprising mass spectroscopy.

74. The method of claim 68 or claim 69 wherein the formation of the complex is detected by a method comprising chromatography.

75. A method for screening a set of exogenous test compounds of unknown biological activity for potential biological activity, said method comprising the steps of selecting a drug-binding substrate exhibiting specific affinity for a drug substance having said biological activity known to derive from specific binding to one or more related endogenous molecules;
contacting said substrate with a test solution comprising said set of test compounds or a subset thereof under conditions known to be conducive to binding of said drug-binding substrate to the drug substance;
separating the test solution from the drug-binding substrate and any test compounds specifically bound to said drug-binding substrate;
separating test compounds specifically bound to said substrate from said substrate and from each other to provide at least one substantially pure test compound;
identifying the test compounds that specifically bind to the drug-binding substrate; and
assaying those identified test compounds for the biological activity.

76. The method of claim 75 wherein the drug-binding substrate is an antibody.

77. The method of claim 75 wherein the drug-binding substrate exhibits specific binding affinity for at least first and second drug substances having the biological activity.

78. The method of claim 75 wherein the drug-binding substrate comprises at least two unique drug-binding substrates which exhibit specific binding affinity for a drug substance having the biological activity.

79. The method of claim 75 wherein the drug-binding substrate is a monoclonal antibody.

80. The method of claim 75 wherein the drug-binding substrate comprises a phage display library.

81. The method of claim 75 wherein the drug-binding substrate comprises a molecularly imprinted polymer

82. The method of claim 75 wherein the contacting of the drug-binding substrate with the test solution, the washing of the drug-binding substrate and the separation of the test compounds that specifically bind to the drug-biding substrate from the drug-binding substrate is carried out by affinity chromatography.

83. The method of claim 75 wherein the step of separating and identifying the test compounds that specifically bind to the drug-binding substrate comprises the step of chromatographically separating said compounds and subjecting same to physicochemical evaluation, structure elucidation or confirmatory testing for binding affinity to a drug-binding substrate.

84. The method of claim 75 wherein the set of test compounds is a natural product comprising an extract of tissue, plant, fungal, microbial or marine origin.

85. A substantially pure test compound having biological activity and prepared and identified in accordance with the method of claim 84.

86. The method of claim 75 wherein the test compounds are members of a combinatorial chemical library.

87. The method of claim 75 wherein a test compound identified to bind specifically to the drug-binding substrate is tested for its capacity for specific binding to the one or more related endogenous molecules.

88. The method of claim 87 wherein each of said monoclonal antibodies forming the drug-binding substrate exhibit specific binding affinity to two or more drug substance having the biological activity.

89. The method of claim 88 wherein the drug-binding substrate comprises a monoclonal antibody raised against a test compound previously identified to exhibit specific binding affinity to a monoclonal antibody exhibiting specific binding affinity to the drug substance.

90. In a method for detection or analysis of specific binding affinity between an endogenous compound known to mediate a biological function, or a derivative or analog thereof, and one or more test compounds in a set of test compounds comprising potential drug substances to identify those test compounds having potential biological activity deriving from specific binding to said endogenous molecule, wherein such method the endogenous compounds or a derivative or analog. thereof is labeled with a detectable label and subjected to capillary electrophoresis in the presence of one or more test compounds, the improvement comprising the step of substituting at least one monoclonal antibody for said endogenous compound or derivative or analog thereof, said monoclonal antibody being selected from those monoclonal antibodies that specifically bind to at least one drug substance known to bind specifically to said endogenous molecule to affect its biological activity and produce a therapeutic effect

91. The improved method of claim 90 wherein the monoclonal antibody is capable of specific binding to at least two drug substances known to specifically bind to said endogenous molecule.

92. The improved method of claim 91 wherein the monoclonal antibody is fluorescently labeled.

93. An anti-drug substance antibody having a detectable label and capable of binding specifically to the drug substance against which it was raised and to at least one other drug substance having the same or similar biological activity.

94. The anti-drug substance antibody of claim 92 wherein the antibody is a monoclonal antibody.

95. The anti-drug substance antibody of claim 93 or claim 94 wherein the label is covalently linked to the antibody.

96. The anti-drug substance antibody of claim 95 wherein the label is a fluorescent label.

97. In a method for screening a set of test compounds to identify those test compounds having potential biological activity, said method comprising the step of measuring or detecting the specific binding of said test compounds to a biologically functional molecule known to be associated with said biological activity or a chemically related derivative or analog thereof, the improvement comprising the step of substituting a monoclonal antibody for said biologically functional molecule, said antibody being selected for its specific binding affinity to a drug substance known to exhibit said biological activity.

98. The improved method of claim 97 wherein the antibody is selected for its specific binding affinity to at least two drug substances known to exhibit said biological activity.

99. The improved method of claim 97 or claim 98 wherein the method measuring or detecting comprises immobilizing the antibody on a sensor chip, contacting the chip with a solution of a test compound and measuring the change in surface optical characteristics of the sensor chip using surface plasmon resonance.

100. The improved method of claim 97 or claim 98 wherein the method of measuring or detecting comprises the step of subjecting the antibody and the test compounds to capillary zone electrophoresis.
